(19) United States
(12) Patent Application Publication
(10) Pub. No.: US 2010/0069620 A1
(43) Pub. Date: Mar. 18, 2010

(54) NOVEL COMPOSITIONS OF CHEMICALLY MODIFIED SMALL INTERFERING RNA

(75) Inventor: Gerald Zon, San Diego, CA (US)

Correspondence Address: ROPES & GRAY LLP, PATENT DOCKETING 39/41, ONE INTERNATIONAL PLACE, BOSTON, MA, 02110-2624, US

(73) Assignee: RXi Pharmaceuticals Corp., Worcester, MA (US)

(21) Appl. No.: 12/227,893

(22) PCT Filed: Jan. 23, 2007
(86) PCT No.: PCT/US2007/060934

§ 371 Date: Nov. 23, 2009

Related U.S. Application Data
(60) Provisional application No. 60/632,682, filed on Dec. 2, 2004.

(51) Int. Cl. C07H 21/02

(52) U.S. Cl. 536/23.1

(57) ABSTRACT

The present invention is directed to compositions comprising chemically modified siRNA that have high specificity by virtue of no or insignificant off-target activity of the sense strand, no or insignificant induction of IFN-like responses, high potency to offset oligonucleotide manufacturing costs, favorable manufacturing chemistry, and effective means of intracellular delivery both in vitro, during target validation and model studies, and in vivo, during animal model studies and clinical trials in humans.

### TECHNICAL FIELD

This invention relates to novel compositions of chemically modified small interfering RNA (“siRNA”) which have utility in the general area of RNA interference (“RNAi”) for either in vitro or in vivo use.

### BACKGROUND OF THE INVENTION

The potency of siRNA as a potential therapeutic agent through RNA interference is important for effective treatment of disease. Research conducted with unmodified siRNA and fully-modified phosphorothioate “PS” antisense oligodeoxynucleotide “AS-ODN” analogs have found that the IC50 of siRNA to be ˜100-fold lower (M. Miyagishi, M. Hayashi, and K. Taira, Antisense and Nucleic Acid Drug Discovery (2003) 13, 1-7, R. Kretschmer-Kazemi Far and G. Sczakiel, Nucleic Acids Res. (2003) 31, 4417-4424 and J. R. Bertrand, et al., Biochem Biophys. Res. Commun. (2002) 296, 1000-1004), while another comparison found ˜1.000-fold lower IC50 for siRNA (A. Grunweller, et al., Nucleic Acids Res. (2003) 31, 3185-3193). However, other studies using optimized siRNA sequences vs. AS-ODNs gapmers (2′-O-methoxyethyl/PS) (T. A. Vickers, et al., J. Biol. Chem. (2003) 278, 7108-7118) obtained comparable activity, as did another investigation with peptide nucleic acids (Y. Liu, et al., Biochemistry (2004) 43, 1921-1927). While the underlying factors for these disparate relative potencies is not known, the multiple examples (M. Miyagishi et al. supra, R. Kretschmer-Kazemi Far et al. supra, J. R. Bertrand et al. supra and A. Grunweller et al. supra) of siRNA having 2-3 orders of magnitude greater activity than AS-ODN has prompted investigation of RNA interference “RNAi” in cases where a promising AS-ODN pharmaceutical has been identified.

Chemical modifications of siRNA to further enhance its effectiveness as a therapeutic agent have been investigated. RNAi activity has been observed in mammalian cells with siRNAs that incorporate PS linkages at the 5′ and 3′ ends, or at alternating linkages, and with 2′-fluoro “F” at all pyrimidines (J. Harborth, et al. (2003) 13, 83-105). Others (F. Czauderna, et al., Nucleic Acids Res. (2003) 31, 2705-2716) found that activity is maintained with alternating 2′-O-methyl “Ome” linkages or several PS and OMe modifications at 5′ and 3′ ends (M. Hemmings-Mieszczak, et al., Nucleic Acids res. (2003) 31, 2117-2126 and M. Amarzguioui, et al., Nucleic Acids Res. (2003) 31, 589-595). Activity has been preserved with more extensive modification with variable numbers of PS, OMe, and locked nucleic modifications (D. A. Braasch, et al., Biochemistry (2003) 42, 7967-7975). Remarkably, in a broad survey of modifications activity was preserved, more or less, even with complete alteration of the sense strand by PS, OMe, or F (Y. L. Chiu and T. M. Rana, RNA (2003) 9, 1034-1048).

A key step identified in the RNAi pathway is assembly of the RNA-induced silencing complex “RISC” which mediates target RNA cleavage through an active form, “RISC*”, that contains only the antisense strand of the siRNA. Inappropriate incorporation of the sense strand of siRNA into RISC* can lead to off-target RNA cleavage at sites homologous to the sense-strand complement (A. L. Jackson, et al., Nature Biotechnol. (2003) 21, 635-637). Recent thermodynamic analyses (D. S. Schwarz, et al., Cell (2003) 115, 199-208 and A. Khvorova, A. Reynolds, and S. D. Jayasena, Cell (2003) 115, 209-216) have led to proposed (A. Khvorova et al. supra) sequence selection rules to favor incorporation of the antisense strand. It was hoped that such asymmetric loading would abrogate sense-strand-mediated off-target cleavage, as well as increase potency due to increased concentration of RISC* loaded with the antisense strand (A. Khvorova et al. supra). More specifically, it was proposed (A. Khvorova et al. supra) that active siRNA should exhibit enhanced flexibility at the 5′ antisense terminus and an overall low internal stability profile, in particular within the 9-14 basepair region of the duplex. It was further suggested (A. Khvorova et al. supra) that altering the chemical or structural nature of the siRNA duplex (introducing mismatches and chemical modifications), which will alter the internal stability profiles to resemble the desirable one, might be a means for optimization of siRNA activity. Preliminary support for thermodynamic manipulation of RNAi activity is found in more recent studies using 3′-end mismatches in novel constructs called “fork-siRNA duplexes” (H. Hohjoh, FEBS Lett. (2004) 557, 193-198). Another recent development in this area is found in advertising information from Dharmacon for siSTABLE™ siRNA, which are said to have a chemically-modified sense strand that reduces off-target effects (see www.dharmacon.com). While the nature of this chemical modification is not divulged, there is a comment in the scientific literature (Q. Ge, et al., Proc. Natl. Acad. Sci. USA (2003) 100, 2718-2723) implying that the sense strand is uniformly modified with OMe substituents. This assumed mode of substitution has been confirmed in a subsequently published patent application by D. Leake et al. assigned to Dharmacon (US 2004/0198640 A1) which states that the presence of 2′-O-methyl modifications are well tolerated on sense but not antisense strands of the siRNA duplex.

In a recent investigation it was observed that in interferon “IFN” mediated activation of the Jak-Stat pathway and global upregulation of IFN-stimulated genes resulted with transfection of siRNA (C. A. Sledz, et al., Nat. Cell. Biol. (2003) 5, 834-839). This effect is mediated by the double-stranded RNA “dsRNA” dependent protein kinase, which is activated by relatively short 21-mer siRNA (C. A. Sledz et al. supra and S. Frantz, Nature Rev. Drug Discovery (2003) 2, 763-764). Abrogation of this limitation has been claimed in a patent application by Sequitur (T. M. Woolf and K. A. Wiederholt, WO 2003/064626 A2) claiming certain “oligomer compositions.” While the detailed nature of these compositions is not clearly specified, they are now offered as Stealth™ RNAi by Invitrogen, Inc. (Carlsbad, Calif.) following its acquisition of Sequitur, Inc. (Natick, Mass.) (see www.invitrogen.com). The aforementioned siSTABLE™ siRNA are similarly claimed by Dharmacon RNA Technologies (Lafayette, Colo.) to abrogate IFN-related or other “cellular toxicity.”

In an attempt to resolve some of the issues regarding the effective use of siRNA as a therapeutic agent Hohjoh (FEBS Lett. (2002) 521, 195-199) used hybrid sense-DNA/antisense-RNA and reported induction of RNAi activity in human cells. This intriguing observation was confirmed by others (J. S. Lamberton and A. T. Christian, Mol. Biotechnol. (2003) 24, 111-120) who additionally found that such DNA/RNA constructs exhibited RNAi activity which was greater in both duration and percent knockdown than that shown by conventional siRNA. One disadvantage of this approach is that unmodified DNA bound to RNA provides a substrate for RNase H(S. T. Crooke, Annu. Rev. Med. (2004) 55, 61-95). Consequently, the unmodified DNA/RNA constructs which have been used (H. Hohjoh et al. supra, J. and S. Lamberton and A. T. Christian supra) to date to induce RNAi are subject to competitive degradation by RNase H which lessens their RNAi potency. Leake et al., supra have also described the suitability of what they call “deoxyribohybrid” type modifications in RNAi where deoxyribohybrids are defined as RNA/DNA hybrid oligonucleotides having deoxy- and ribo-entities in an oligonucleotide, for example, in a sequence of alternating deoxy- and ribonucleotides. They further specify that “[i]ₜ is important in the design of these kinds of oligos to keep the size of continuous DNA/RNA duplex stretches shorter than 5 nucleotides to avoid the induction of RNase H activity.”

Another disadvantage of the aforementioned molecular design specifications by Hohjoh, supra, Christian, supra, and Leake et al., supra is that the sense-DNA strand in unmodified or chemically modified DNA/RNA can lead to undesired off-target effects by binding to complementary or partially homologous mRNA and then cleavage by RNase H (S. T. Crooke, supra) and/or blockage of transcription.

Small interfering RNA, like antisense oligonucleotides are postulated to treat a number of diseases. Advances are being made in siRNA delivery as is evidenced by the commercial availability of a wide variety of in vitro cellular transfection agents (e.g. www.mirusbio.com) and citations in review articles dealing with in vivo delivery (e.g. “Systemic delivery of synthetic siRNAs,” S. Mouldy and D. R. Sorensen, Methods in Molecular Biology (2004), 252, 515-522).

At present there is a need for successful siRNA compounds that have high specificity by virtue of no or insignificant off-target activity of the sense strand, no or insignificant induction of IFN-like responses, high potency to offset oligonucleotide manufacturing costs, favorable manufacturing chemistry, and effective means of intracellular delivery both in vitro, during target validation and model studies, and especially in vivo, during animal model studies and clinical trials in humans.

### BRIEF DESCRIPTION OF THE DRAWING

FIG. 1 depicts the thermal melting curves for various 19-mer HER-2 conjugate oligonucleotides. Oligonucleotide 1 (see Table 1) is depicted by filled squares. Oligonucleotide 2 is depicted by filled triangles. Oligonucleotide 3 id depicted by filled circles.

FIG. 2 depicts the effect of various oligonucleotides on cell survival.

FIG. 3 depicts the induction of apoptosis in human MDA-MB-435 breast carcinoma tumors treated with various oligonucleotides. The lane indicated by an “H” is oligonucleotide 2 in Table 1.

### DETAILED DESCRIPTION OF THE INVENTION

One aspect of the present invention is directed to siRNA hybrid duplexes “siRNA analogs” that have reduced susceptibility to RNase H cleavage, decreased off-target effects resulting from the binding of the sense-DNA strand to partially homologous mRNA which increases susceptibility to RNaseH, and increased rate of duplex disassociation thereby increasing potency. Another aspect of the present invention is compatibility with sequence designs that favor RISC-loading of the antisense strand and disfavor RISC-loading of the sense strand, i.e. asymmetric loading.

In one aspect of the present invention a DNA/RNA duplex is provided comprising an antisense RNA “R” strand (wherein each “R” represents a ribonucleotide) and a sense strand comprising OMe “O” substituents in the central region of the sense strand (wherein each “O” represents a 2′-O-methylribonucleotide) with DNA “D” flanks (wherein each “D” represents a deoxyribonucleotide). The “sense” and the “antisense” strands, by definition are complementary to one another.

In one embodiment, the length of each strand of the DNA/RNA duplex is between 12 and 30 nucleotide pairs.

In another embodiment, the DNA/RNA duplex additionally comprises single-stranded overhangs at the 5′ end, the 3′ end or both the 5′ and 3′ end. The overhangs can range in length from 1 to 6 nucleotides.

In yet another embodiment, the DNA/RNA duplex is blunt-ended.

In another embodiment, each of the DNA flanks on the sense strand is independently 3 to 10 nucleotides in length.

In another embodiment, each of the DNA flanks on the sense strand is the same length.

In another embodiment the 2′-O-methylribonucleotide central region of the sense strand is 3 to 10 nucleotides in length.

In one particular embodiment the DNA/RNA duplex is provided in a “5-9-5” 19-mer design shown below. In this construction the number of OMe residues is minimized and the helical-DNA/RNA “footprint” is too small to readily accommodate RNase H. The “5-9-5” and 19-mer design as examples of the invention is not meant to be limiting but it is intended to exemplify the use of degradable flanking DNA sequences (which could use unmodified or modified DNA bases).

| | |
|---|---|
| Sense | D D D D D O O O O O O O O O D D D D D |
| Antisense | R R R R R R R R R R R R R R R R R R R |

This configuration also reduces the off-target effects resulting from the sense strand following dissociation because the 3′ and 5′ DNA-ends of the construct are susceptible to degradation by exonucleases leaving a relatively short OMe fragment with a low Tm and, therefore, low hybridization potential toward inhibition of translation of off-target mRNA.

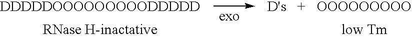

Dissociation is important in efficient release of the antisense RNA strand. One method of dissociation results from RNA helicase A, which unwinds DNA/RNA duplexes (K. Zhou, et al., Nucleic Acids Res. (2003) 31, 2253-226 and refs. cited therein). Another method involves incorporation of single-stranded RNA into RISC* which mediates target RNA cleavage (J. Martinez, et al., Cell (2002) 110, 563-574 and T. Holen, M. Amarzguioui, E. Babaie, and H. Prydz, Nucleic Acids Res. (2003) 31, 2401-2407).

In another embodiment of the present invention the rate of duplex-dissociation may be increased by incorporation of multiple deoxyinosine “1” units in the sense strand effectively lowering the Tm. The I-units may be located within the 5′ DNA flank, the 3′ DNA flank, or both the 5′ and the 3′ DNA flanks of the sense strand.

In one embodiment, the number of I-units incorporated into any DNA flank is from 1 to 6 nucleotides.

In another embodiment of the present invention this incorporation of I-units may be located at only one of the two ends of the sense strand (i.e., incorporated into only one of the DNA flanks). When I-units are thus incorporated in only the 3′ flank of the sense strand they induce biased Tm lowering such that RISC-loading preferentially occurs with the antisense strand (i.e., asymmetric loading) and may therefore lead to increased levels of RNAi activity or specificity or both.

An additional advantage of such 3′ and/or 5′ incorporation of I-units is the possibility of a second mechanism of degradation of the released sense strand by endonuclease V. This enzyme is known to be present in human cells and causes removal of deoxyinosine moieties from single stranded DNA (A. Moe, et al., Nucleic Acids Res. (2003) 31, 3893-3900).

At the same time, inhibition of ribonuclease degradation of the released antisense RNA strand can be achieved by use of 2-3 phosphorothioate (PS) linkages (indicated by R′) at the 5′ and 3′ ends of the antisense RNA strand. Thus, in another embodiment, the DNA/RNA hybrid of this invention has at least one 2-3 phosphorothioate (PS) linkage present at either the 3′ end or the 5′ end of the antisense strand. In one embodiment, between one and six 2-3 phosphorothioate (PS) linkages are present at one or both ends of the antisense strand.

In one embodiment, the DNA/RNA hybrid of this invention has the structure indicated below. This hybrid advantageously incorporates 2-3 phosphorothioate (PS) linkages, and I units to increase stability and increase duplex dissociation.

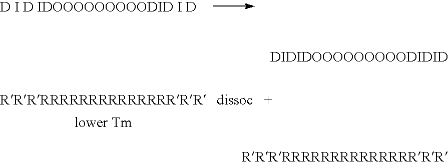

wherein each D is a deoxyribonucleotide; each I is a deoxyinosine nucleotide; each R is a ribonucleotide; each R′ represents 2-3 phosphorothioate (PS) linkage; and each O is a 2′-O-methylribonucleotide.

In another embodiment DNA/RNA duplex stabilization may be important for dissociation. In such a case incorporation of C5-propynyl pyrimidines into the DNA sense strand will increase binding to RNA.

### Examples

All uses of RNAi employing siRNAs are predicated on the selection of an effective target sequence that is complementary to the antisense strand of the siRNA. The field of siRNA-mediated RNAi has progressed rapidly over the past several years and, as a result, the skilled artisan will be familiar the availability of sequence design guidelines for siRNA. Non-limiting examples of these guidelines are given in K. Ui-Tei, et al., Nucleic Acids Res. (2004) 32, 936-948 and pertinent refs. cited therein as well as A. Khvorova et al. in patent application no.: WO 2004045543. These readily available guidelines are therefore used to select a desired number of gene-specific candidate sequences against which the corresponding chemically modified siRNA analogs of the present invention are synthesized using commercially available reagents and procedures that are known to skilled artisans in the field of oligonucleotide synthesis, or which can be readily obtained from any one of a number of custom oligonucleotide vendors.

Detailed guidelines are also available for in vitro uses of siRNAs (e.g., S. M. Elbashir, et al., Methods (2004) 26, 199-213; Y. Dorsett and T. Tuschl, Nature Reviews (2004) 3, 318-329; M. Sohail in “Gene Silencing by RNA Interference: Technology and Application,” CRC Press LLC, Boca Raton Fla. (to be published in 2005). RNAi has been rapidly adopted as a general method for inhibiting gene expression in most laboratory organisms. Libraries of RNA reagents have been used to perform genome-wide reverse genetic screens in both model organisms and mammalian cells. B. Lehner, et al., Briefings in Functional Genomics & Proteomics (2004) 3, 68-83.

The nucleotide sequences of the oligonucleotides used in this study are shown in Table 1.

TABLE 1

Sequences of 19-mer Anti-HER-2 and Control HER-2 Cognate
Oligonucleotides^{a}

| | | | 5′->3′ Sense (top) |
|---|---|---|---|
| No. | Name | Abbreviation | 3′->5′ Antisense (bottom) |
| 1 | HER-2 duplex 3 | HD | UCUCUGCGGUGGUUGGCAU |
| | | | AGAGACGCCACCAACCGUA |
| 2 | HER-2 hybrid 3 | HH | TCTCTGCGGTGGTTGGCAT |
| | | | AGAGACGCCACCAACCGUA |
| 3 | HER-2 modified hybrid | mH | TITITgcggugguuGICIT |
| | 3 | | AGAGACGCCACCAACCGUA |
| 4 | Control HER-2 hybrid | CH | TTCTCCGAACGTGTCACGT |
| | | | AAGAGGCUUGCACUGAGCA |
| 5 | Control HER-2 | CmH | TICICcgaacguguCICIT |
| | modified hybrid | | AAGAGGCUUGCACAGUGCA |

^{a}RNA = capital letters in normal font; DNA = capital letters in bold font; 2′OMe = lowercase letters in normal font.

The oligonucleotides were chemically synthesized using commercial phosphoramidites (Glen Research, Sterling, Va. and Pierce Chemical, Rockland, Ill.) and ethyl thiotetrazole (AIC) on an 8909 Expedite synthesizer (Applied Biosystems, Foster City, Calif.) at a 15-μmol scale following manufacturers' recommended protocols. After standard deprotection procedures, the DNA and mixed DNA/2′OMe/DNA oligonucleotides were purified by reverse-phase HPLC. The RNA oligonucleotides were deprotected and desilylated using standard procedures, desalted using LH-20 columns (Amersham Biosciences), and then purified by preparative PAGE. All oligonucleotides were precipitated from ethanol as sodium salts and quantified by conventional UV260 calculations. Purity of the oligonucleotides was determined by analytical PAGE and HPLC analyse and was estimated to be >90-95% in all cases. Identity of the oligonucleotides was confirmed bymass spectrometry (HT Labs, San Diego). All oligonucleotides were synthesized with 5′-hydroxyl groups, except when stated otherwise.

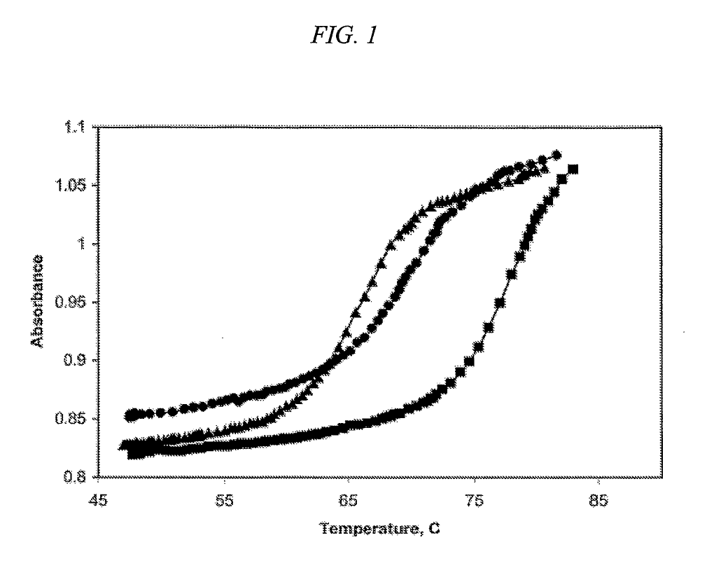

Tm measurements were performed on a Beckman DU640B Spectrophotometer equipped with a water-jacketed UV-cell holder. A water-circulating thermostat provided linear increase of the temperature (1-2° C./min) inside the UV-cell from room temperature to ˜80° C. Temperature was controlled by a ThermologR Themistor thermometer. The concentration of each oligonucleotide strand was 2.6 μM. Samples were dissolved in 10 mM sodium phosphate buffer containing 100 mM sodium chloride and 1 mM EDTA, pH 7.4. Before UV measurements, the samples were heated to 90° C. for 5 min, then slowly cooled to room temperature and transferred to a 1-mL UV-cell. Tm values for the resultant duplexes were determined from the melting curve as the temperature of the maximum of the first derivative (ΔA/ΔT) vs. T, where A is absorbance as defined above and T is temperature (° C.). The Tm curves and Tm values are given in FIG. 1.

A mixture of single-stranded antisense oligonucleotide (1 μmol) and its single-stranded cognate oligonucleotide (1 μmol) in water (10 mL) was prepared in a 15-mL screw-cap plastic tube. The capped tube was placed in a beaker containing 100 mL of boiling water and then allowed to slowly cool to room temperature. To ensure that each pair of oligonucleotides formed a duplex, a 5-μL aliquot of the annealed mixture was added to 15 μL of loading buffer (1×TBE in 50% glycerol). After 10-60 min incubation at room temperature, the mixture was subjected to analytical non-denaturing PAGE together with each single strand loaded in a separate lane as a size marker. During the run the temperature of the gel was maintained below 40° C. to prevent thermal melting (see FIG. 1). The mixture was stored frozen at −20° C.

One to one molar ratios of each single-stranded antisense and cognate sense oligonucleotide were annealed. Cationic liposome (dioleoyltrimethylammonium phosphate [DOTAP] and dioleoylphosphatidylethanolamine [DOPE] [Avanti Polar Lipids, Alabaster, Ala.]) was prepared at a 1:1 molar ratio by ethanol injection as described in Xu, L. et al., Molecular Medicine 2001, 7, 723-734. The anti-transferrin receptor single-chain antibody fragment (TfRscFv) was mixed with the liposome at a ratio of 1:30 (w/w). The siRNA molecules were subsequently added to the admixture at a ratio of 1 μg siRNA to 7 nmol liposome, followed by sizing and confirmation of nanosize particle distributions of the final immunoliposome formulations by dynamic light scattering with a Malvern Zetasizer 3000 HS (Malvern, Worcestershire, UK).

In vitro transfections were performed as follows. 4×103 PANC-1 cells were plated/well of a 96-well plate. After 24 h, the cells were transfected with TfRscFv-LipA complexes, prepared as described above, containing either the hybrid (HH), control hybrid (CH), modified hybrid (mH), or control modified hybrid (CmH) compounds 2-5, respectively. The concentration of siRNA analog varied from 0.4 to 250 nM. The optimized (for activity vs. toxicity) ratio of LipA to siRNA analog was 7 to 1 (nmol:μg). A conventional colorimetric cell-viability assay using 2,3-bis(2-methoxy-4-nitro-5-sulfophenyl)-5-[(phenyl-amino)carbonyl]-2H-tetrazolium hydroxide (XTT) [32] was performed 48 h after transfection. Error bars represent triplicate measurements. All experiments were independently reproduced at least twice and provided substantially the same results (data not given).

For the in vivo studies, human breast carcinoma tumors were induced in female athymic nude (NCR nu/nu) mice by subcutaneous inoculation of 6×106 MDA-MB-Y35 cells suspended in Matrigel® collogen borement membrane (BD Biosciences, Bedford, Mass.).

Mice bearing tumors of at least 100 mm³ were treated with 3 mg/kg anti-HER-2 hybrid (HH), control hybrid (CH), anti-HER-2 modified hybrid (mH), or control modified hybrid (CmH) compounds 2-5, respectively, encapsulated in TfRscFv-LipA. The complex was prepared as described above using the ratio of LipA to siRNA of 7 to 1 (nmol:μg) Treatment was by i.v. injection three times over 24 h. Mice were sacrificed 46 h after the first injection and 20 h after the last injection. Forty micrograms of total protein isolated from each tumor was electrophoretically fractionated using a Criterion Precast 4-20% gradient gel transferred to nylon membrane and then immunostained for expression levels of HER-2 (rabbit polyclonal antibody C-18; Santa Cruz Biotechnology, Inc., Santa Cruz, Calif.), phosphorylated AKT (pAKT) (mouse monoclonal antibody Ser 473; Cell Signaling Technology™, Beverly, Mass.), phosphorylated mitogen-activated protein kinase (pMAPK) (mouse monoclonal antibody, Thr 202/Tyr 204, E10; Cell Signaling Technology™), cleaved caspase-3 (rabbit polyclonal antibody Asp175; Cell Signaling Technology™), antiapoptotic protein BCL-2 (rabbit polyclonal antibody N-19; Santa Cruz Biotechnology, Inc.), and the housekeeping gene glyceraldehyde-3-phosphate dehydrogenase (GAPDH) (rabbit polyclonal antibody; Trevigen, Inc., Gaithersburg, Md.).

Compound 1 shown in Table 1 is a 19-mer, blunt-ended version of an RNA/RNA duplex (HD) that had been previously reported as a 21-mer with 3′ d(TT) overhangs to have RNAi activity against HER-2. This truncated RNA/RNA duplex (HD), compound 1, provided a reference Tm of 79.3±0.3° C. for the expected melting transition from double- to single-stranded species (FIG. 1). The ˜14° C. decrease in Tm to 65.7±1.0° C. found for hybrid (HH) compound 2, wherein the RNA sense strand of compound 1 is replaced by DNA, was consistent with the well-known generalization that DNA/RNA hybridization is less stable than RNA/RNA. The ˜4.0° C. increase in Tm to 69.1±0.5° C. for modified hybrid (mH) compound 3, wherein the DNA sense strand in compound 2 is replaced by a chimeric “5/9/5” motif of DNA/2′OMe/DNA, was consistent with the well-known generalization that introduction of 2′OMe moieties into oligonucleotides increases Tm. Although we did not characterize the corresponding control HER-2 compounds 4 and 5, we estimate that they have roughly comparable Tm values, relative to 2 and 3, respectively, based on the presence of 10 vs. 11 GC-basepairs. In any case, these Tm measurements for compounds 1-3 confirmed that the shortened 19-mer RNA/RNA siRNA (HD) and its DNA/RNA hybrid (HH) had GC content adequate for encapsulation and intracellular delivery of largely double-stranded species, which also applied to DNA/2′OMe/DNA modified hybrid (mH) even though 4 dI residues were incorporated.

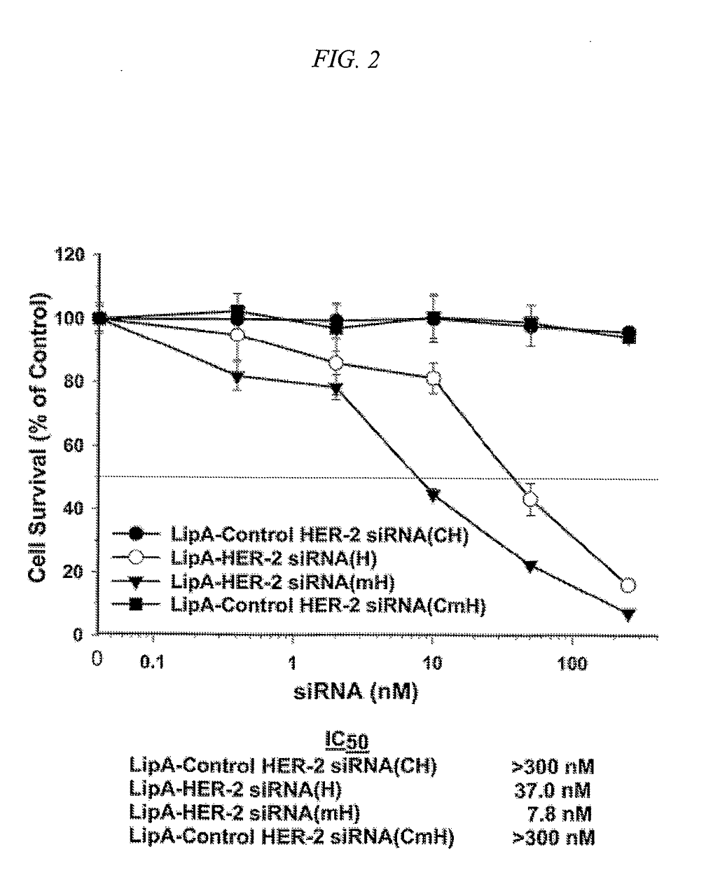

As indicated by the results shown in FIG. 2, treatment of PANC-1 cells with a TfRscFv-targeted immunoliposome formulation of anti-HER-2 hybrid (HH), compound 2, led to significant killing of this pancreatic cancer cell line. This effect was dose-dependent over the studied range of 0.4 to 250 nM and had an IC50 value (the dose resulting in 50% survival) of 37.0 nM. In another experiment (data not given), this hybrid (HH) had an IC50 value in a similar range (68 nM), whereas compound 1, which is the corresponding RNA/RNA duplex (HD), gave an IC50=100 nM. This slightly greater potency of the hybrid (HH) vs. duplex (HD) composition was consistently reproduced in multiple independent experiments, as was the inactivity (IC50>300 nM) of control hybrid (CH), compound 4 (FIG. 2), and control duplex (data not given). Increased potency of RNAi upon this type of sense strand RNA replacement with DNA has been previously reported. However, chemical modification of sense strand DNA as embodied in modified hybrid (mH), compound 3, afforded even significantly greater potency, namely, IC50=7.8 nM (FIG. 2).

Sequence specificity was supported by the fact that corresponding control modified hybrid (CmH), compound 5, was inactive (IC50>300 nM). Additional control experiments (data not given) using chemically 5′-phosphorylated versions of compounds 2 and 3 led to essentially unchanged IC50 values.

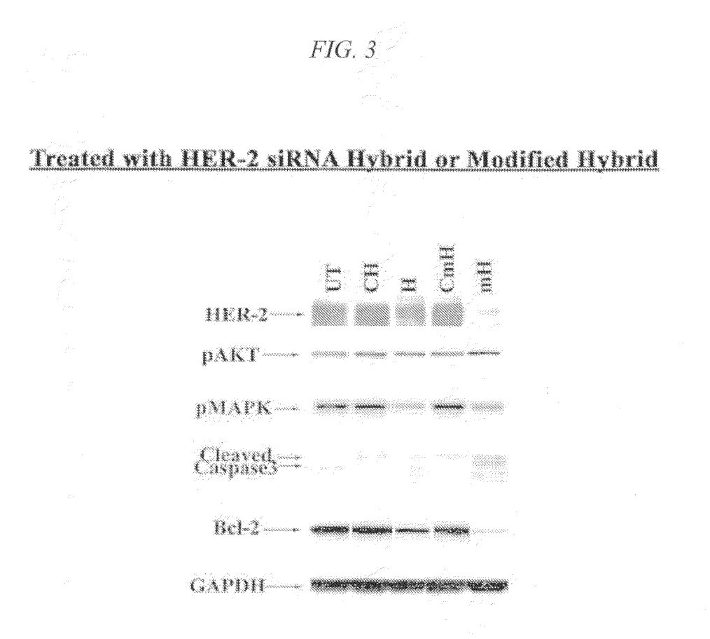

In vivo studies employing a mouse xenograft model of human breast cancer (derived from MDA-MB-435 cells) allowed assessment of the effect of these different siRNA analogues (all directed against HER-2) on the level of expression of selected components in the HER-2 signal transduction pathway in tumors. Immunostaining of electrophoretically separated, tumor-derived proteins shown in FIG. 3 indicated that, following equivalent, repeated i.v. dosing with oligonucleotides at 3 mg/kg, modified hybrid (mH, lane 5), compound 3, induced greater reduction of HER-2, relative to hybrid (HH, lane 3), compound 2. In contrast, HER-2 levels in corresponding Controls (CH, lane 2 and CmH, lane 4), compounds 4 and 5, respectively, were comparable to that for the untreated control (UT, lane 1) sample. For all of these samples, levels of the housekeeping gene, glyceraldehyde-3-phosphate dehydrogenase (GAPDH), were essentially the same, indicating equal protein loading. Of the other proteins that were analyzed, phosphorylated AKT (pAKT) appeared to be largely unchanged, whereas levels of phosphorylated mitogen-activated protein kinase (pMAPK) and the antiapoptotic Bcl-2 proteins were decreased by treatment with HH and mH. The presence of cleaved caspase-3, which is a hallmark of apoptosis, was particularly evident in tumor tissue following treatment with mH vs. HH, as was the reduction of Bcl-2. Consistent with mH-mediated RNAi of HER-2 leading to such changes in cleaved caspase-3 and Bcl-2, these proteins were essentially unchanged upon treatment with controls CmH or CH vs. no treatment. These qualitatively different in vivo effects of mH vs. HH on the HER-2 protein target and downstream apoptosis-related proteins are likely due to the greater RNAi-potency of mH vs. HH that was initially evidenced in vitro by IC50 values associated with cancer cell viability.

We claim:

1. A DNA/RNA duplex comprising:
a) an antisense RNA strand of between 12 and 30 nucleotides;
b) a sense strand of between 12 and 30 nucleotides comprising:
i) a central region having between 3 and 10 2′-O-methylribonucleotides;
ii) two regions flanking and bound to said central region, each of said flanking
regions independently consisting of between 3 and 10 deoxyribonucleotides,
wherein said sense strand and said antisense strand are complementary to one another.

2. The DNA/RNA duplex of claim 1 having the formula:

| | |
|---|---|
| Sense | D D D D D O O O O O O O O O D D D D D |
| Antisense | R R R R R R R R R R R R R R R R R R R |

wherein, each D is a deoxyribonucleotide; each O is a 2′-O-methylribonucleotide; and each R is a ribonucleotide.

3. The DNA/RNA duplex of claim 2, wherein the terminal 1 and 6 ribonculeotides at one or both ends of the antisense strand are bound to an adjacent ribonucleotide through a 2-3 phosphorothioate (PS) linkage.

4. The DNA/RNA duplex of claim 2, wherein between 1 and 5 deoxyribonucleotides in any flanking region of the sense strand are deoxyinosine.

5. The DNA/RNA duplex of claim 1 having the formula:

| | |
|---|---|
| Sense: | D I D I D O O O O O O O O O D I D I D |
| Antisense: | R′ R′ R′ R R R R R R R R R R R R R R′ R′ R′ |

wherein, each D is a deoxyribonucleotide; each O is a 2′-O-methylribonucleotide; each R is a ribonucleotide; each I is deoxyinosine; and each R′ is bound to an adjacent ribonucleotide through a 2-3 phosphorothioate (PS) linkage.
